(19) United States
(12) Patent Application Publication
(10) Pub. No.: US 2004/0005583 A1
(43) Pub. Date: Jan. 8, 2004

(54) Method for analysing nucleic acids

(76) Inventors: Bruno De Vandiere, Toulouse (FR); Herve Crespeau, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/343,510

(22) PCT Filed: Jul. 20, 2001
(86) PCT No.: PCT/FR01/02372

(30) Foreign Application Priority Data
Aug. 1, 2000 (FR) 00/10108

(51) Int. Cl. C12Q 1/68

(52) U.S. Cl. 435/6

(57) ABSTRACT

The present invention concerns compositions and methods for analyzing, separating or sequencing nucleic acids. More particularly, it concerns methods for analyzing, separating or sequencing nucleic acids by electrophoresis, in particular capillary, more preferably gel-free. The invention also concerns methods for treating nucleic acids, or separating nucleic acids, applicable to rapid and efficient sequencing. The invention further concerns products, compositions and kits for implementing said methods. The invention consists particularly in the use of a positively charged carrier molecule, enabling to efficiently separate nucleic acids in solution. The invention is useful for analyzing, separating or sequencing nucleic acids of different type and origin, in the field of experimentation, diagnosis, medicine and the like, in particular de novo sequencing and genotyping.

The present invention concerns compositions and methods for analyzing, separating or sequencing nucleic acids. More particularly, it concerns methods for analyzing, separating or sequencing nucleic acids by electrophoresis, in particular capillary, more preferably gel-free. The invention also concerns methods for treating nucleic acids, or separating nucleic acids, applicable to rapid and efficient sequencing. The invention further concerns products, compositions and kits for implementing said methods. The invention is useful for analyzing, separating or sequencing nucleic acids of different type and origin, in the field of experimentation, diagnosis, medicine and the like, in particular de novo sequencing and genotyping.

The methods and tools for sequencing, analyzing or separating nucleic acids have many applications, for instance in genome analysis, gene research, identification of polymorphisms (particularly SNP), etc. Current methods for sequencing DNA comprise essentially three main steps, namely (i) the sequencing reaction (or chemistry), (ii) separation and (iii) detection.

The sequencing step or reaction consists mainly in producing copies of the nucleic acid to be sequenced, copies which are randomly terminated by incorporation of a chain-terminating nucleotide (typically a dideoxynucleotide). The principle and conditions of this reaction are described for example in Sanger et al. (ref. 1). Typically, the sequencing reaction is carried out on a sample of the nucleic acid to be sequenced, in the presence of a nucleotide primer and a suitable quantity of chain-terminating nucleotides A, C, T or G. The sequencing reaction may be carried out in combined manner by using four differently labelled chain-terminating nucleotides A, C, T or G, or in parallel, by using in each of these reactions one of the four chain-terminating nucleotides A, C, T or G and a differentially labelled nucleotide primer, the products obtained in these four reactions then being pooled. The product of the sequencing reaction therefore comprises a mixture of nucleic acids of different lengths, whose 3′ ends represent each of the bases of the molecule to be sequenced.

The separation step generally consists in separating the species present in the product of the sequencing reaction, so as to determine the sequence of the starting nucleic acid. Generally, this separation is carried out on the basis of the charge and/or mass of each species. In the methods described in the prior art, this separation is mainly carried out by electrophoresis in cross-linked gels or viscous media (usually polyacrylamide). The presence of the gel slows down the migration of the species, and therefore allows resolution of the separation induced by application of an electric field.

The species that are present may be detected or analyzed by classical electrophoretic methods, according in particular to the type of label used (radioactive, fluorescent, etc.). For instance, the products of the sequencing reaction are typically labelled by using labelled chain-terminating nucleotides, each species then carrying an identifiable labelled terminal nucleotide.

The methods of sequencing (or analysis) described in the prior art nevertheless have drawbacks and limitations. In particular, the separation step is frequently limiting, and does not allow an analysis (or sequencing) of very long nucleic acids, or with sufficient rapidity or sensitivity.

In order to improve the efficiency of the separation step, it has been proposed to have recourse to capillary electrophoresis, which makes it possible to work at a high speed and in an automated manner. In particular, capillary electrophoresis allows the use of strong electric fields (200 to 500 V/cm, as compared to 40 with conventional electrophoresis) and automated gel pouring and loading of the nucleic acid samples on the gel. However, these methods have come up against considerable obstacles. For example, it is essential to use highly viscous gels, which are difficult to fill into the capillaries due to their viscosity and the interactions of the gel with the capillary wall, and induce ageing of the gel-filled capillary, which must be changed after a certain number of separations. In an attempt to remedy this problem, different laboratories seek to modify the composition of the gels, so as to obtain good resolution with a low viscosity, allowing rapid filling of the capillary and, if possible, avoiding preliminary anti-electroosmotic surface treatment of the capillary. However, no gel that meets these requirements is currently available. Another approach put forth to try to address this problem consists in grafting nucleic acids on neutral molecules, acting to slow down DNA molecules in liquid medium (ref. 12, 14). Such an approach based on the use of neutral molecules with the aim of slowing migration is also mentioned in U.S. Pat. No. 5,108,568, US 5,624,800 and US 5,470,705 and in application WO00/43547. However, this method does not satisfactorily resolve the problems noted hereinabove and hereinbelow. Furthermore, the read length (resolution) allowed by all these prior methods is approximately 600 to 1100 nucleotides, under optimal conditions, due to the compromise necessary between speed, field and sensitivity (ref. 6, 7).

Other approaches to try to improve separation consist in miniaturized electrophoresis by means of microarrays. For instance, advances in the micro-machining of different materials have led to envisioning micro-fabricated microarrays (microchip technology). However, while this approach produces arrays of reduced size, which can be incorporated into more complex detection systems, it does not resolve the problems related to the gel and so far has not afforded superior performance (ref. 8, 9, 10).

Thus there is a need for improved methods for analyzing nucleic acids, especially for separating nucleic acids, in particular in the context of a sequencing reaction. The present invention now proposes a new, sensitive and efficient concept of nucleic acid separation, which can be automated, by which to analyze or sequence very long nucleic acids on supports as varied as slab gel electrophoresis or capillary electrophoresis (capillary support or chips).

More particularly, the present invention describes a method for analyzing or separating nucleic acids that can be used in gel-free medium.

The present invention concerns in particular a method for analyzing or separating nucleic acids using a positively charged carrier molecule.

The present invention shows preferably that it is possible to achieve good resolution of nucleic acids, without gel, based not on simple frictional forces but on a combination thereof with an added electrical charge.

The present invention therefore proposes a new concept for separating nucleic acids contained in a sample, based on using a positively charged carrier molecule (“drag”). This new approach runs counter to what was taken for granted by those skilled in the art, namely, that since the DNA molecule has a global negative charge, the migration of the sequencing products can only occur in the direction cathode (−) → anode (+). Indeed, this bias has guided all the current approaches for separating nucleic acids described in the prior art. Now, under the action of the electric field, large-sized DNA molecules tend to move along the electric field lines, and in which case have a more or less identical apparent size, rendering any method of separation futile. Furthermore, due to the constant charge/mass ratio, solely the use of suitable separating media (cross-linked or micellar gels) can resolve sequence fragments to within one base, on molecules composed exclusively of DNA.

The present invention now proposes a new strategy, consisting in modifying the charge of the nucleic acids, in order to orient their migration differently and offer better resolution. More particularly, the invention proposes for the first time to treat the nucleic acids to be analyzed so as to couple them to a positively charged carrier molecule (FIG. 1). The presence of this positive charge (Q) reverses the global charge of the object (complex) to be separated, and thus the direction of migration. For this reason, the hereinabove described phenomenon observed with long DNA fragments is strongly attenuated, and such approach makes it possible to envision sequencing nucleic acids more than 2000 bases long, for example up to 5000 bases. In addition, according to the present invention, it is the nucleic acid molecule that slows the migration of the positively charged carrier molecule and not the contrary. For this reason, the smallest fragments are analyzed first and the longest last, the sequence obtained therefore being straightaway in the right orientation, in contrast to the systems of the prior art. Furthermore, the mass of the carrier molecule brings about an improved resolution of the method through frictional forces and bypasses the need to use cross-linked or gelified media.

One object of the present invention therefore consists more specifically in a method for separating nucleic acids by electrophoresis, comprising (i) a step of contacting the nucleic acids with a sufficient quantity of a positively charged carrier molecule in conditions suitable for allowing the formation of complexes between the carrier molecule and all or part of the nucleic acids, the complexes formed being composed essentially of one carrier molecule and one nucleic acid molecule, and (ii) separating the complexes formed by electrophoresis.

Another object of the invention consists in a method for analyzing a nucleic acid sample, wherein it comprises (i) contacting the sample with a sufficient quantity of a positively charged carrier molecule in conditions suitable for allowing the formation of complexes between the carrier molecule and all or part of the nucleic acids in the sample, the complexes formed being composed essentially of one carrier molecule and one nucleic acid molecule, and (ii) analyzing the complexes formed by electrophoresis.

A further object of the invention is a method for sequencing a nucleic acid, wherein it comprises:

(a) a nucleic acid sequencing reaction in the presence of four chain-terminating nucleotides, possibly differently labelled,

(b) contacting all or part of the product of reaction (a) with a sufficient quantity of a positively charged carrier molecule in conditions suitable for allowing the formation of complexes between the carrier molecule and all or part of the nucleic acids present in said product of reaction (a), the complexes formed being composed essentially of one carrier molecule and one nucleic acid molecule, and

(c) separating the complexes formed by electrophoresis, allowing the sequence of the nucleic acid to be determined.

As described hereinbelow, in the above methods, the nucleotides and/or the carrier molecule are advantageously labelled to enable analysis of the sample. Thus, in the case of a sequencing method, the chain-terminating nucleotides are advantageously labelled differently, or the nucleotide primer or the carrier molecule.

The invention may be used for analyzing nucleic acids by different types of electrophoresis. For instance, when not defined more specifically, the term electrophoresis in the context of the invention refers to any electrophoretic method, such as for example capillary electrophoresis (in capillary tubes or microarrays (“micro-chips”)), “slab gel” electrophoresis (in cross-linked gels), gel-free electrophoresis (“free-flow” electrophoresis), magnetophoresis (electrophoresis with application of a magnetic field), etc.

The invention further provides a method for treating a nucleic acid sample, comprising contacting the sample with a sufficient quantity of a positively charged carrier molecule, in conditions suitable for allowing the formation of complexes between the carrier molecule and all or part of the nucleic acids in the sample, the complexes formed being composed mainly of one carrier molecule and one nucleic acid molecule.

The method of the invention may be used for separating, analyzing, treating or sequencing any type of nucleic acid. For instance, it may be DNA or RNA, single stranded or double stranded, particularly cDNA, gDNA, synthetic or semisynthetic or recombinant DNA, mRNA, etc. Typically, the nucleic acid is single stranded, preferably single stranded DNA. The nucleic acids may be of natural, synthetic, semisynthetic, recombinant origin, etc. In particular they may be nucleic acids of mammalian, eukaryotic, prokaryotic, plant, viral origin, etc. By way of illustration, they may be nucleic acids of animal or human origin, particularly human or animal DNA or RNA, in particular single stranded. They may also be viral or bacterial nucleic acids, particularly from pathogenic organisms. The nucleic acid may be of variable length, linear, circular, supercoiled, etc. It may consist of DNA fragments, amplification products, mixtures of nucleic aids of different origin and/or type, etc. Preferably, the method of the invention is used to analyze or separate a nucleic acid sample comprising at least 100 different species, more generally at least 500 different species. It is understood that the method of the invention is not restricted to a particular type of nucleic acid or sample.

As indicated hereinabove, an important feature of the present invention consists in the use of a positively charged carrier molecule, to which each species (or a part thereof) of the sample is coupled. Such carrier molecule enables the separation of nucleic acids in liquid solutions, with good efficiency and high sensitivity. Furthermore, the use of a carrier molecule according to the invention allows separation of the species in order of increasing size.

In a preferred manner, to implement the invention the carrier molecule satisfies the following conditions:

it has a high molecular weight, and/or

it has a positive electrical charge greater than the negative charge of the longest nucleic acid molecule to be analyzed (e.g., separated, sequenced).

More preferably, the positively charged carrier molecule has a molecular weight greater than or equal to 100,000 daltons, preferably 200,000 daltons, more preferably 300,000 daltons.

Furthermore, in a preferred manner, the positively charged carrier molecule has a positive electrical charge such that the ratio R of the positive charge of the carrier molecule to the negative charge of the longest nucleic acid molecule to be analyzed is greater than 1, preferably greater than or equal to approximately 1.2, more preferably greater than or equal to 1.5, even more preferably greater than or equal to 2. This ratio may be determined according to conventional methods known to those skilled in the art. To this end, if necessary or desired, the positive charge of the carrier molecule may be increased by artificial means, for example by polymerization or functionalization, or yet by any modification of the physico-chemical parameters of the electrophoresis.

In addition, the invention is advantageously implemented by using a mainly monodisperse carrier molecule composition, i.e. composed of homogeneous species, preferably forming a single band on electrophoretic migration. The monodisperse character may be achieved by a purification of carrier molecules. Preferably, the carrier molecule forms a single isofocalization band under non-denaturing conditions, allowing to differentiate pH variations of 0.02 units. It is nonetheless understood that a variability (or heterogeneity) is tolerated.

In an especially advantageous manner, the carrier molecule is able to form a complex with a nucleic acid of the sample, a complex composed mainly of one carrier molecule and one nucleic acid molecule. In fact, to enable the resolution of the nucleic acids, it is important that the nucleic acid species present in the medium remain separate, and therefore that the complexes formed contain only a single nucleic acid molecule. However, it is understood that the formation, in a low proportion (generally less than 10%, more preferably less than 5%) of complexes containing several nucleic acid molecules coupled with a carrier molecule cannot be entirely eliminated, without this lowering the efficiency of the method. The interaction between the carrier molecule and the nucleic acid may be covalent or non-covalent. In particular it may be a physical or chemical bond, particularly electrostatic, hydrogen, van der Waals, etc. Preferably, the interaction (and the formation of complexes) is ensured by the presence on the carrier molecule of a functional group A. Such functional group enables the formation of a specific complex between said molecule and a nucleic acid molecule. The functional group A may be chosen for example from among avidin, a receptor, a ligand, an antibody, or a fragment or derivative thereof retaining a specificity, a bifunctional reagent, etc. or any reactive group ensuring the formation of a bond or a specific interaction with a defined partner. More preferably, the carrier molecule harbors a single functional group A. The term “single” indicates that each carrier molecule bears only a single molecule of functional group A. This characteristic is advantageous because it allows the formation of defined complexes between a single carrier molecule and a single nucleic acid molecule.

In a particular embodiment, the carrier molecule contains an avidin or avidin-derived functional group.

The functional group A may be naturally present or artificially introduced into the carrier molecule, according to its origin and type. As shown in the examples, the functional group may be introduced on the molecule in particular through a physical or chemical bond, directly or by means of a reagent or intermediate linker (FIG. 2). By way of example, the functional group may be introduced by means of a bridging agent, able to interact (physically or chemically) with the carrier molecule on the one hand and the functional group A on the other hand. Such an agent may be chosen in particular from the maleimide groups, particularly compounds MBS (ref. 22311), sulfo-MBS (ref. 22312), SMPB (ref. 22416), sulfo-SMPB (ref. 22317), GMBS (ref. 22309), sulfo-GMBS (ref. 22324), EMCS (ref. 22308) or sulfo-EMCS (ref. 22307) available through the Pierce company. Typically, these bridging agents may be used to couple any functional group A to a carrier molecule containing at least one reactive SH group.

For this reason, in a more preferred embodiment of the invention, the carrier molecule contains at least one reactive SH group.

It is understood that any other coupling method and/or any other bridging agent may be used within the scope of the present invention.

According to an especially advantageous embodiment, the carrier molecule has:

a high molecular weight, preferably greater than or equal to 100,000 daltons, preferably 200,000 daltons, more preferably 300,000 daltons,

a positive electrical charge such that the complex formed with the longest nucleic acid molecule to be analyzed remains positive, and

a single functional group A, chosen preferably from among avidin, a receptor, a ligand, an antibody, or a fragment or derivative thereof retaining a specificity, or any reactive group allowing to form a bond or a specific interaction with a defined partner. Furthermore, as shown hereinbelow, the carrier molecule may also be labelled, to allow its detection. In this context, several labels may be envisioned, of the enzymatic, radioactive, fluorescent, luminescent, biological type, etc. Depending on the conditions of implementation of the methods of the invention, labelling of the carrier molecule can allow to detect the nucleic acids and to carry out the sequence, or the genotyping. A preferred label is the fluorescent label.

The carrier molecule may be of different type and origin. For example, it may be a synthetic or natural molecule of biological, chemical, mineral or organic origin. In a particular embodiment, the carrier molecule is mainly a polypeptide or of polypeptidic origin (i.e., comprising a chain of several amino acids). As noted hereinabove, it may be a polypeptide of natural or synthetic origin, for example recombinant or synthesized by means of synthesizers for instance. The polypeptide may also be modified, for example chemically, enzymatically, etc., to confer additional properties or to improve its properties (or its electrical charge).

In the case of a recombinant polypeptide, it may be produced in any suitable cellular organism, particularly in bacteria (without glycosylation), yeasts, mammalian cells, insect cells, etc.

When it is a synthetic molecule, it may be produced by synthesis in several fragments, which may be assembled and possibly modified according to conventional methods.

Specific examples of polypeptide molecules which may be used in the present invention are hemocyanin, hemoglobin, albumin, EIF3 P110 (from yeast) or the beta chain of KW antigen (from rickettsia). A specific and preferred example is hemocyanin, particularly from limulus.

Hemocyanin has a size (molecular weight) comprised between 500,000 and 3.5 million daltons depending on its origin and polymerization; it is composed of 6 to 8 polypeptide chains and has a net positive charge in electrophoresis buffers. Moreover, the alpha chain (F176I) contains a single cysteine having a free SH group (FIG. 3). This chain (or polymerized forms thereof) represent a more particular example of carrier molecule according to the invention.

It is understood that other molecules may be chosen by those skilled in the art, which satisfy the hereinabove parameters and which may be used within the context of the invention.

As noted hereinabove, the method of the invention comprises placing the nucleic acids in contact with the carrier molecule (monodisperse), in conditions suitable for allowing the formation of complexes between such molecule and all or part of the nucleic acids to be analyzed or separated.

To ensure higher efficiency of the method, it is important that the largest number (or percentage) of nucleic acid molecules present in the sample be engaged in a complex. Furthermore, it is equally important in order to obtain better resolution, that the complexes contain mainly a single carrier molecule and a single nucleic acid molecule. Finally, it is also preferable that the carrier molecule be monodisperse. Under such conditions, for placing in contact, one advantageously uses a molar ratio of carrier molecule/nucleic acid greater than or equal to 1. In fact it is especially advantageous to proceed in the presence of an excess of carrier molecule, so that the highest proportion of nucleic acid species will be engaged in a complex and can therefore be analyzed. It is understood that the present invention may be implemented without it being necessary that all the nucleic acids in the sample be engaged in a complex, and without each complex formed containing only a single nucleic acid. However, the greater the extent to which all these conditions are met, the higher the quality of the analysis (or length of the sequence determined).

As indicated hereinabove, the coupling may be covalent or non-covalent, physical or chemical. In a preferred embodiment, the coupling is carried out by means of a functional group A present on the carrier molecule and a functional group B present on the nucleic acids. In fact it is desirable that the coupling can be accomplished in a simple, efficient and specific manner.

For this reason, in a preferred embodiment, the nucleic acids to be treated contain a functional group B, able to interact specifically with the functional group A. The functional group B is therefore composed of a partner of functional group A, i.e. for example, biotin, a ligand, a receptor, an antigen (or epitope), or a fragment or derivative thereof retaining a specificity, or any reactive group ensuring the formation of a specific bond (or interaction) with functional group A. Specific interaction or bond designates an interaction or bond between two partners displaying a respective affinity.

In a specific embodiment, the nucleic acids contain a functional group B composed of a biotin molecule, and the coupling is carried out through an interaction of the type avidin-biotin.

In another embodiment, the nucleic acids contain a functional group B composed of an antigen or antigen fragment, and the coupling is carried out by a reaction of the type antigen-antibody.

In a further embodiment, the coupling is carried out by specific hybridization using a particular oligonucleotide which can be grafted onto the carrier molecule. Such oligonucleotide may be specific of a nucleic acid region, and allow a complex to be formed by specific hybridization.

A preferred example is based on the use of the (strept)avidin-biotin system. Thus, nucleic acid molecules may be functionalized to carry a single biotin molecule, and the carrier molecule contains an avidin functional group. Contacting the two leads to the formation of monodisperse complexes through an avidin-biotin interaction. Each complex can then be separated by electrophoresis, according to the invention.

The nucleic acids may be functionalized in different ways. For example, they may be functionalized after their synthesis, by terminal chemical modification according to known methods. They may also be functionalized during their synthesis, for example during an amplification reaction. In this respect, in the case of a sequencing reaction, the production of copies of the nucleic acid to be sequenced may be accomplished by amplification using a labelled oligonucleotide primer (for example biotinylated). In such case, each copy synthesized (from the primer) will carry said label. The results obtained show that the use of a biotinylated primer does not alter the sequencing reaction, and allows to generate a population of nucleic acids labelled by a functional group B.

In this respect, a particular object of the present application consists in a method for sequencing a nucleic acid, wherein it comprises:

(a) a sequencing reaction of the nucleic acid in the presence of four differentially labelled chain-terminating nucleotides and a biotinylated primer,

(b) contacting all or part of the product of reaction (a) with a sufficient quantity of a positively charged carrier molecule containing an avidin group, so as to form complexes between the carrier molecule and all or part of the nucleic acids present in said product of reaction (a), the complexes formed being composed mainly of one carrier molecule and one nucleic acid molecule, and

(c) separating the complexes formed by electrophoresis, allowing the sequence of the nucleic acid to be determined.

In another variant of the methods of the invention, sequencing step (a) comprises four sequencing reactions carried out in parallel each with a different chain-terminating nucleotide chosen from among A, C, T or G and a primer having a functional group B and differentially labelled (i.e. with a different label for each of the four reactions).

In a further variant of the methods of the invention, sequencing step (a) comprises four sequencing reactions carried out in parallel each with a different chain-terminating nucleotide chosen from among A, C, T or G and a primer having a functional group B, and, in step (b), each product of the four reactions is contacted in parallel with the differentially labelled carrier molecule (and preferably containing a functional group A). In this embodiment, the analysis may then be performed in parallel or after having pooled the different complexes formed.

The carrier molecule and the nucleic acids may be placed in contact in any suitable medium and apparatus. Buffer solutions are preferably used, for example conventional electrophoresis buffers. Even more preferably, solutions are used in which the carrier molecule has a net positive charge. The placing in contact may take place prior to or concurrently with the introduction of the components into the separation device. The separation is then generally carried out in the same buffer suitable for electrophoresis, allowing the complexes formed to go into suspension.

One advantage of the present invention is that it offers the possibility of carrying out the separation in gel-free medium. This feature is especially advantageous because it allows the sequencing of long nucleic acids (longer than 1500 bases), in conditions that are easy to implement, and at a low cost. Of course, it is also possible to add to the separation medium cross-linking components, such as polyacrylamide, in proportions that can be adjusted by those skilled in the art.

In a preferred manner, the complexes are separated by capillary electrophoresis. In such case, different commercial apparatuses may be used, such as capillaries having a diameter less than or equal to approximately 200 μm, more preferably less than or equal to approximately 100 μm, typically comprised between approximately 10 and approximately 50 μm, or by capillary electrophoresis systems performed on solid substrates, wherein the capillary is essentially a channel etched on this same substrate.

As noted hereinabove, in a preferred embodiment, the carrier molecule-nucleic acid complexes are labelled, so as to enable their analysis. The labelling of the species to be separated may be done in different ways. For example, it may be a labelling of the nucleic acids or of the carrier molecule. Furthermore, in the case of labelling of the nucleic acids, it may be an end-labelling (“terminal dye”) or a primer labelling (“primer dye”), or yet a labelling of the carrier molecule.

In the case of terminal dye, the labelling is achieved during the sequencing reaction through the use of labelled chain-terminating nucleotides. In this embodiment, the sequencing reaction may be carried out in a combined manner with the different types of chain-terminating nucleotides.

In the case of labelling of the primer or the carrier molecule, several (generally four) sequencing reactions are conducted in parallel in the presence of one of the four chain-terminating nucleotides, either with four sets of differentially labelled primers, or in the presence of the same primer, the reactions products subsequently being contacted in parallel with four sets of differentially labelled carrier molecules.

The labels used are preferably fluorescent, radioactive, enzymatic, luminescent, chemical labels, etc. Typically, four different fluorochromes are used to label (i) the chain-terminating nucleotides or (ii) the primers or (iii) the carrier molecule.

The complexes resolved by the methods of the invention may then be analyzed by conventional methods. Furthermore, the methods of the invention may be implemented by using different types of devices or apparatuses, particularly sequencers, known to those skilled in the art and/or commercially available.

For instance, “automated” sequencers may advantageously be used, which in fact provide automated reading of the electrophoresis. Such sequencers first became available in the late 1980s. See in particular L. Hood et al., ref. 2, concerning a sequencer that operates with fluorescent primers; ref. 3 concerning a sequencer that runs with fluorescent dideoxynucleotides; and Ansorge et al. (ref. 4).

More recent apparatuses make use of “slab gel” detection systems. A specific example comprises sequencers from the Perkin Elmer company. In such sequencers, DNA fluorescence is detected by scanning the electrophoresis gel. Color separation was initially provided by filters placed between the gel and the detector (one color was detected per scan, and four scans were needed to detect the four colors). In more recent instruments (Sequenceur 377), a grating diffracts the light emitted by the gel, and a CCD camera collects the various wavelengths already spatially separated. This increases the detection speed four-fold, and it has been possible to similarly increase the electrophoresis speed. These commercially available Perkin Elmer sequencers are therefore capable of detecting four fluorophores in a same electrophoresis lane.

Other sequencers sold by Pharmacia or LiCor may be used. In particular, the Pharmacia sequencers contain no moving parts. The gel is illuminated by one laser on the slide, and photodiodes are placed opposite each electrophoresis lane. This detection system has high sensitivity. The LiCor sequencer has the particular feature of operating in the infrared (6). It works by scanning, but the very low background noise in the infrared makes it a very sensitive apparatus.

One model of this sequencer operates with gels 60 cm in length, which makes it possible to increase the length of the sequences being read to 800 to 1000 nucleotides.

It is understood that the invention is not restricted to a particular device or apparatus, and that it may be implemented by any useful means.

Another object of the invention consists in a composition comprising a nucleic acid coupled to a positively charged carrier molecule, such as defined hereinabove.

A further feature of the invention concerns compositions comprising a mixture of nucleic acids complexed with a positively charged carrier molecule, each complex comprising essentially one carrier molecule and one nucleic acid.

More preferably, in the compositions provided for in the invention, the nucleic acid or nucleic acids are labelled, for example by radioactive, fluorescent, luminescent, enzymatic, chemical labels, etc.

Moreover, according to a further embodiment, in the compositions of the invention, the carrier molecule is labelled, for example by radioactive, fluorescent, luminescent, enzymatic, chemical labels, etc.

Another object of the invention is based on the use of a positively charge carrier molecule for separating nucleic acids by electrophoresis, in particular capillary. It is more particularly a carrier molecule such as defined hereinabove, advantageously containing a functional group A and/or a label.

The invention equally concerns kits for analyzing nucleic acids, comprising a positively charged carrier molecule, having a high molecular weight and a functional group, and, optionally:

a capillary whose internal diameter is suited to ensure the separation of the nucleic acid molecules, and/or

the products required for preparing the buffer in which the separations will be carried out, and/or

the instrumental conditions of separation, such as the electrophoretic current, voltage, capillary length, type of capillary, etc.

The preferred kits in the context of the invention are intended for sequencing nucleic acids, and comprise a positively charged carrier molecule, having a high molecular weight and a functional group A and a nucleotide primer for the sequencing reaction, containing a functional group B.

In the kits of the invention, the carrier molecule is preferably defined as hereinabove. Such kits may furthermore contain several sets of primers or carrier molecules, differentially labelled.

As indicated hereinabove, the invention is useful for analyzing, separating or sequencing any nucleic acid (or nucleic acid sample), in the field of experimentation, diagnosis, therapeutics, pharmacogenomics, genotyping and the like.

Other features and advantages of the present invention will become apparent in the following examples, which are given for purposes of illustration and not by way of limitation.

### LEGENDS TO FIGURES

FIG. 1: Principle of coupling with a carrier molecule (“DNA-Drag”).

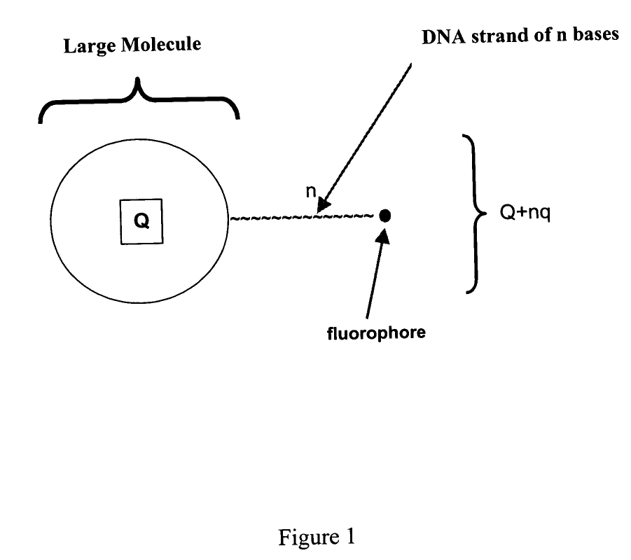

FIG. 2: Coupling of the nucleic acid with the carrier molecule by means of an avidin-biotin system.

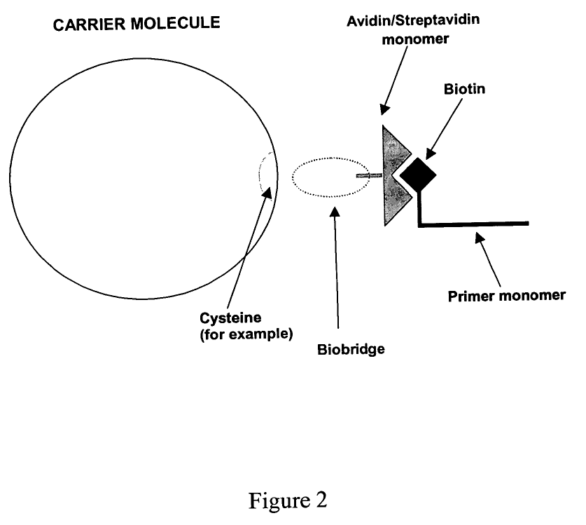

FIG. 3: Molecular structure of hemocyanin.

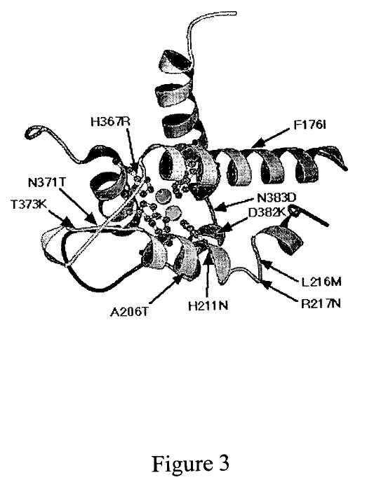

### EXAMPLES

In this example, hemocynanin obtained from Sigma (ref. H1757) is used as the carrier molecule. This molecule is derived from Limulus polyphemus (horseshoe crab) by purification on a DEAE-Sephadex column. The molecule is repurified by acrylamide gel electrophoresis, to obtain a monodisperse product.

Avidin is used as the functional group A. The avidin used is deglycosylated (Fluka ref. 11367). This molecule is extracted from egg white. After reconstitution, the molecule is repurified on an acrylamide gel.

The two electrophoretically purified molecules are then coupled by using a bi-functional bridging agent: Sulfo-BMS (m-maleimidobenzoyl-N-succinimide ester) from Pierce Biochemical (ref. 22312). The avidin is then made monomeric by using the method described elsewhere (ref. 13).

Coupling is carried out as follows:

Sulfo-BMS is dissolved in distilled water at 10 mM final concentration, immediately before the reaction. A coupling buffer is prepared containing: 83 mM sodium phosphate buffer pH 7.2 and 900 mM NaCl (PBS). It is also possible to use HEPES, carbonate/bicarbonate, or borate buffer. The buffer preferably does not contain an amine or thiol group. The proteins are extensively dialysed against this buffer if they are already dissolved, otherwise they are dissolved in this buffer in the presence of 10 mM EDTA.

100 μl of a 2 mg/ml solution of sulfo-BMS are added to 200 μl of a 10 mg/ml solution of repurified deglycosylated avidin. This is allowed to stand at room temperature for one hour. The 300 μl are then desalted on a column. A 1.5 ml fraction is collected from the column after elution of the first two ml. Five hundred microliters of a 4 mg/ml hemocyanin solution are then added and the solution is allowed to stand at room temperature for two hours. The reaction is stopped by adding 50 μl of 1X TBE buffer. The carrier molecule (containing functional group A) is preferably purified by exclusion or affinity chromatography. The molecule is then ready for use in the nucleic acid separation.

The sequencing reaction is conducted according to the protocols recommended by the different manufacturers. At this stage, a “dye terminator” reaction is performed, by using a biotinylated primer and four chain-terminating nucleotides labelled with different fluorophores. Once the sequencing reaction is terminated, the carrier molecule is added to the tube, in a suitable quantity, and the solution is mixed by gentle shaking. The resulting mixture is then loaded in the sequencer for analysis.

A “dye primer” sequencing reaction may also be used. For this, four sequencing reactions are conducted in parallel with each of the chain-terminating nucleotides, and four coupling reactions are done, using the carrier molecule labelled by four different fluorophores. The reactions can then be combined and the resulting mixture loaded in the sequencer for analysis.

### REFERENCES

1 F. Sanger, S. Nicklen and A. R. Coulson. DNA sequencing with chainterminating inhibitors. Proc. Natl. Acad. Sci. USA, 1997; 74: 5463-5467.

2 L. M. Smith, J. Z. Sanders, R. J. Kaiser, P. Hughes, C. Dodd, C. R. Connell, C. Heiner, S. B. H. Kent and L. E. Hood. Fluorescence detection in automated DNA sequence analysis. Nature, 1986; 321: 674-678.

3 J. M. Prober, G. L. Trainor, R. J. Dam, F. W. Hobbs, C. W. Robertson, R. J. Zagursky, A. J. Cocuzza, M. A. Jensen and K. Baumeister. A system for rapid DNA sequencing with fluorescent chain-terminating dideoxynucleotides. Science, 1987; 238: 336-341.

4 W. Ansorge, B. S. Sproat, J. Stegemann and C. Schwager. A non-radioactive automated method for DNA sequence determination. J. Biochem. Biophys. Methods, 1986; 13: 315-23.

5 L. R. Middendorf, J. C. Bruce, R. C. Bruce, R. D. Eckles, D. L. Grone, S. C. Roemer, G. D. Sloniker, D. L. Steffens, S. L. Sutter, J. A. Brumbaugh and G. Patonay. Continuous, on-line DNA sequencing using a versatile infrared laser scanner/electrophoresis apparatus. Electrophoresis, 1992; 13: 487-494.

6 J. A. Luckey, T. B. Norris and L. M. Smith. Analysis of resolution in DNA sequencing by capillary gel electrophoresis. J. Phys. Chem., 1993; 97: 3067-3075.

7 J. A. Luckey and L. M. Smith. Optimization of electric field strength for DNA sequencing in capillary gel electrophoresis. Anal. Chem. 1993; 65: 2841-50.

8 D. J. Harrison, K. Fluri, K. Seiler, Z. Fan, C. S. Effenhauser and A. Manz. Micromachining a miniaturized capillary electrophoresis-based chemical analysis system on a chip. Science, 1993; 261: 895-897.

9 A. T. Wooley and R. A. Mathies. Ultra-high-speed DNA fragment separations using microfabricated capillary array electrophoresis chips. Proc. NatI. Acad. Sci. USA, 1994; 91: 11348-11352.

10 A. T. Wooley and R. A. Mathies. Ultra-high-speed DNA sequencing using capillary electrophoresis chips. Anal. Chem., 1995; 67: 3676-3680.

11 Zahia Djouadi. Séquençage de l'ADN par l'électrophorèse: étude des effets thermiques sur la migration. Physics-Biology Interface Group, Nuclear Physics Institute of Orsay, THESIS presented on Jul. 12, 2000.

12 Hongji Ren, Achim E. Karger, Frank Oaks, Steve Menchen, Gary W. Slater and Guy Drouin. Separating DNA sequencing fragments without a sieving matrix. Electrophoresis, 1999; 20 No. 12: 2501-2509.

13 Michael Green and E John Toms. The properties of subunits of avidin coupled to sepharose. Biochem. J., 1973; 133: 687-700.

14 CurChristoph Heller et al. Free-solution electrophoresis of DNA. J. Chromatography A, 1998; 806: 113-121.

1. A method for separating nucleic acids by electrophoresis, comprising (i) a step of contacting the nucleic acids with a sufficient quantity of a positively charged carrier molecule under conditions suitable for allowing the formation of complexes between the carrier molecule and the nucleic acids or a fraction thereof, the complexes formed being positively charged and composed essentially of one carrier molecule and one nucleic acid molecule, and (ii) separating the complexes formed by electrophoresis.

2. A method for analyzing a nucleic acid sample, wherein said method comprises (i) contacting the sample with a sufficient quantity of a positively charged carrier molecule under conditions suitable for allowing the formation of complexes between the carrier molecule and the nucleic acid(s) in the sample, the complexes formed being positively charged and composed essentially of one carrier molecule and one nucleic acid molecule, and (ii) analyzing the complexes formed by electrophoresis.

3. A method for sequencing a nucleic acid, wherein said method comprises:
(a) a sequencing reaction of the nucleic acid in the presence of four differently labelled chain-terminating nucleotides,
(b) contacting all or part of the product of reaction (a) with a sufficient quantity of a positively charged carrier molecule under conditions suitable for allowing the formation of complexes between the carrier molecule and the nucleic acid(s) present in said product of reaction (a), the complexes formed being positively charged and composed essentially of one carrier molecule and one nucleic acid molecule, and
(c) separating the complexes formed by electrophoresis, allowing the sequence of the nucleic acid to be determined.

4. A method for treating a nucleic acid sample, comprising contacting the sample with a sufficient quantity of a positively charged carrier molecule, under conditions suitable for allowing the formation of complexes between the carrier molecule and the nucleic acid(s) in the sample, the complexes formed being positively charged and composed essentially of one carrier molecule and one nucleic acid molecule.

5. Method according to any one of claims 1 to 4, wherein the positively charged carrier molecule has a high molecular weight and a positive electrical charge greater than the negative charge of the largest nucleic acid molecule to be analyzed.

6. Method according to claim 5, wherein the positively charged carrier molecule has a molecular weight greater than or equal to 100,000 daltons.

7. Method according to claim 5 or 6, wherein the carrier molecule is essentially polypeptidic.

8. Method according to claim 7, wherein the positively charged carrier molecule is chosen from the group of hemocyanin, hemoglobin, albumin and EIF3 P110.

9. Method according to claim 7, wherein the positively charged carrier molecule is of recombinant or synthetic origin.

10. Method according to any one of the previous claims, wherein the positively charged carrier molecule contains a functional group A.

11. Method according to claim 10, wherein the functional group A is chosen from the group of avidin, a receptor, a ligand, an antibody and a reactive group ensuring a specific coupling with a defined partner.

12. Method according to any one of the previous claims, wherein the carrier molecule has:
a high molecular weight, preferably greater than or equal to 100,000 daltons, preferably 200,000 daltons, more preferably 300,000 daltons,
a positive electrical charge chosen so that the complex formed with the longest nucleic acid molecule to be analyzed remains positive, and
a single functional group A, chosen preferably from among avidin, a receptor, a ligand, an antibody, or a fragment or derivative thereof retaining a specificity, or any reactive group ensuring the formation of a bond or a specific interaction with a defined partner.

13. Method according to any one of the previous claims, wherein the nucleic acids contain a functional group B.

14. Method according to claim 13, wherein the functional group B is a specific partner of the functional group A present on the carrier molecule.

15. Method according to any one of the previous claims, wherein the positively charged carrier molecule contains an avidin group and wherein the nucleic acids contain a biotin group.

16. Method according to any one of the previous claims, wherein the electrophoresis is a capillary electrophoresis, carried out preferably in capillaries with a diameter less than approximately 200 μm, or in channels etched on a substrate.

17. Method according to any one of claims 1 to 16, wherein the nucleic acid is a single stranded nucleic acid, preferably a single stranded DNA.

18. Composition comprising a nucleic acid coupled to a positively charged carrier molecule, the global charge of the complex thus formed being positive.

19. Composition according to claim 18, comprising a mixture of nucleic acids complexed to a positively charged carrier molecule, each complex comprising essentially one carrier molecule and one nucleic acid.

20. Composition according to claim 18 or 19, wherein the nucleic acid(s) is or are labelled.

21. Composition according to claim 18 or 19, wherein the carrier molecule is labelled.

22. Utilization of a positively charged carrier molecule for separating nucleic acids by electrophoresis, by inversion of the global charge of the complex formed between the carrier molecule and the nucleic acid to be separated.

23. Kit for analyzing nucleic acids, comprising a positively charged carrier molecule having a molecular weight above 100 000 daltons and a functional group.

24. Kit for sequencing nucleic acids, wherein it comprises a positively charged carrier molecule, having a high molecular weight and a functional group A and a nucleotide primer for sequencing reaction, comprising a functional group B.
